(19) United States
(12) Patent Application Publication
(10) Pub. No.: US 2004/0013752 A1
(43) Pub. Date: Jan. 22, 2004

(54) Buccal and sublingual mucosally absorbed herbal compositions for relieving nicotine withdrawal symptoms and craving for nicotine and nicotine containing substances

(76) Inventor: Philip E. Wolfson, San Anselmo, CA (US)

Correspondence Address: Richard C. Litman, LITMAN LAW OFFICES, LTD., P.O. Box 15035, Arlington, VA, 22215, US

(21) Appl. No.: 10/611,911

(22) Filed: Jul. 3, 2003

Related U.S. Application Data
(60) Provisional application No. 60/394,157, filed on Jul. 5, 2002.

(51) Int. Cl. A61K 35/78

(52) U.S. Cl. 424/751

(57) ABSTRACT

Compositions useful in relieving withdrawal symptoms and acute and later craving in nicotine dependent/habituated persons are provided that includes an herbal component. That herbal component provides one or more naturally occurring nicotine agonists, and zero to trace amounts of nicotine (unless explicitly formulated with some designated amount of nicotine plus the herbal component). The compositions are explicitly designed to disperse and have th active principle(s) absorbed in the oral cavity.

### CROSS-REFERENCE TO RELATED APPLICATION

This application claims the benefit of U.S. Provisional Patent Application Serial No. 60/394,157, filed Jul. 5, 2002.

### BACKGROUND OF THE INVENTION

1. Field of the Invention

The present invention generally relates to compositions useful in relieving the symptoms of nicotine withdrawal and craving for nicotine as contained in any and all of the myriad products that contain nicotine, such as cigarettes, cigars, smoking tobacco, chewing tobacco, and products that contain nicotine as a chemical, this in nicotine habituated persons who are abstaining from or reducing nicotine intake; and more particularly the invention relates to compositions that include an herbal component which provides multiple nicotine agonists, one of which may be anabasine, but contain little or no nicotine; and which are designed to be absorbed through the mucosal tissues of the oral cavity.

2. Description of Related Art

Using 1996 data, the prevalence of cigarette smoking in the United States among adults was about 27% or 55 million people. Each year some 30% of smokers try to quit, but only about 10% are successful. Th fficacy rat for formal cessation programs, defined as abstinence of one-year follow-up, is between 20 and 40% of those enrolled. The most telling fact is that the majority of smokers who are successful in quitting tobacco have done so on their own. In the past ten years, 47.5% of persons attempting to quit smoking on their own were successful compared to 23.6% of those who used smoking cessation programs to quit.

There have been many therapies and pharmacologic agents used to assist in smoking cessation. Nicotine delivered through gum, transdermal patches, and nasal sprays in declining dosages over time have been the principal pharmacologic strategies, i.e., a withdrawal over time minus the tar of actual cigarettes.

More recently the anti-depressant bupropion has been reintroduced in a long acting twice-a-day preparation for smoking cessation. The anxiolytic buspirone has been suggested for use as an adjunct for the treatment of nicotine addiction. These preparations are costly, may have undesirable side effects and require prescriptions and medical supervision.

There have been several herbal preparations suggested for smoking cessation. A lobella-based preparation was withdrawn because of FDA concerns sparked by toxicity reports from human use. U.S. Pat. No. 4,817,640, issued Apr. 4, 1989 to Summers, describes herbal chew and snuff products, which are said to proximate the texture, taste, and organoleptic sensation of a snuff or chew composition. The herbs are selected from dandelion, papaya, dock or sorrel, sunflower, calendula, nasturtium, mallow, chicory, corn silk, and mixtures th reof. In addition, clover is suggest d for us , with red clover being the preferred major component for the snuff composition.

Among other smoking cessation products have been chewing gums that include pure anabasine in salt form. Thus, Russian Patent No. 1,268,141, published Nov. 7, 1986, describes an anti-nicotine gum formed by mixing an aqueous anabasine-HCl solution into syrup, and formulating further with a bas and sugar. U.S. Pat. No. 4,971,079, issued Nov. 20, 1990 to Talapin et al., describes another chewing gum carrier where an alkaloid, preferably anabasine hydrochloride, is coupled via a cation exchange group to a biological absorbable polymeric vehicle, and this coupled composition is then formulat d in a chewing gum.

U.S. Pat No. 5,942,244, issued Aug. 24, 1999 to Friedman et al., describes tablet formulations for local and slow release of herbal medication into the oral cavity of a subject.

Anabasine and other alkaloids, such as anatabine, are structurally similar to nicotine, and are believed to substitute for nicotine (as agonists) at nicotine receptor sites.

### SUMMARY OF TBE INVENTION

The present invention consists of compositions useful in relieving craving in nicotine habituated and dependent persons who are voluntarily abstaining from or reducing nicotine consumption. The invention consists of an herb or an h rbal extract providing one or more naturally occurring nicotine agonists, at least ne of th nicotine agonists being anabasine in an amount of at l ast about 0.1 (1/10^{th} of one percent) weight percent of the herb or herbal extract, the herb or herbal extract having from about 0 weight percent nicotine to trace levels of nicotine therein. The composition further includes carriers (e.g. solid or liquid) for the herb or herbal extract some of which will facilitate dispersion and absorption of these alkaloids—anabasine, anatabine, etc—through the mucosal lining of the oral cavity and into the circulation.

A preferred combination of nicotine agonists is anabasine and anatabine provided by flowers, dried leaves, stems, and/or roots, particularly of the Nicotiana glauca plant, or an herbal extract thereof. Suitable carriers for oral mucosal absorption include gums or binders (particularly for chewing gum formulations), sucking candies, syrups, oral films, intra-oral sprays, sub-lingual liquids, oral fast dissolving tablets for sub-lingual dispersion, micro-emulsions, sublingual buccal effervescents, trans-mucosal delivery systems, lozenge formulations, and any and all delivery systems having the potential for enabling in one form or another the oral absorption of the active principles, such as anabasine, etc.

### DETAILED DESCRIPTION OF THE PREFERRED EMBODIMENTS

Broadly, compositions of this invention are suitably formulated for oral mucosal absorption (e.g., chewing gums, sucking candies, syrups, oral films, intra- ral sprays, sub-lingual liquids, oral fast dissolving tablets for sub-lingual dispersion, micro-emulsions, sublingual buccal effervescents, trans-mucosal deliv ry systems, lozenge formulations, and the like). Regardless of the particular f rm, th compositions consist ssentially f an h rbal component that is d rived from a plant or mixtures of plants having a quantity of naturally occurring alkaloid agonists of nicotine such as anabasine, but with little or no nicotine. Among th plants from which the herbal component may be obtained are, for exampl , Medicago sativa, Lupinus formosus, Solonum carolinense, Aniba coto, Zinnia elegans, Sophora pachycarpa, Verbascum songaricum, Priestleya elliptica, Priestleya tomentosa, Haloxylon persicum, Haloxylon sallcornicum, and Nicotiana glauca. Some species include quantities of both anabasine and anatabine and zero to small amounts of nicotine, such as N. glauca and N. debneyl (with anabasine predominating).

A particularly preferred plant for obtaining the herbal component is N. glauca (commonly called “tree tobacco”). This plant grows wildly in the western United States. It has been medicinally used as an analgesic poultice applied externally. Anabasine is the most prominent of the nicotine like alkaloids in N. glauca leaves and other parts of the plant.

The herbal component of this invention will usually be provided by ( r derived from) plant foilage (leaves and stems), although plant roots may also be used, since the concentrations of naturally occurring nicotine agonists may vary in the different parts of each respective plant. The herbal component may be prepared as dried plant parts, or any of a variety of extracts therefrom. Herbal extracts are extracts of plant materials, such as, for example, a tinctur of botonical materials, which typically are prepared by contacting botanical material with a solvent (British Herbal Pharmacopoeia, Peter R. Bradley, Ed., British Herbal Medicine Association, 1983; and British Herbal Compendium, Peter R. Bradley, ed., British H rbal Medicine Association, 1992). Th solvent, for example, can be aqueous or organic, or a combination thereof. Acceptable organic solvents include, but are not limited to, glycerin, propylene glycol, ethanol or other alcohols, hexane, methylene chloride or a combination thereof. The most preferred solvents are hydro alcoholic solvents as defined in British Herbal Pharmacopoeia and Compendium. Other extraction methods may be used—such as super-critical carbon dioxide, liquid nitrogen, fractionation, wiped film drying, etc

Since a smoking cessation program may begin by gradual cessation of nicotine usage, followed by more complete, or by complete cessation of nicotine usage, inventive compositions may be formulated that have som nicotine (albeit in quantities substantially less than the naturally occurring nicotine agonist alkaloids, often in a program of diminishing amounts over time of these substances, these substances being absorbed through the oral mucosa, the purpose of this being to diminish or even eliminate the symptoms that occur as a result of nicotine withdrawal, including acute and later cravings for nicotine in its myriad forms. Or the same or similar program may be provided using only the naturally occurring nicotine agonists, without the presence of any quantity of nicotine; or only trace amounts of nicotine that are not substantially activ because of their very small concentrations in the herbal materials being used.

Use of herbs or herbal extracts in accordance with this invention may provide a complex mixture of ingredients. Since an agonist stimulates th receptor by stabilizing an active confirmation, and this stabilization can be achieved in many different ways depending upon the chemical nature of the ligand and on the structure of th receptor,th combination of agonists provided from a source of complex ingredients, such as the suitable herbs or herbal extracts of this invention, may achieve a stabilizing function through multiple interactions at different parts of the target receptor, thus reducing nicotine withdrawal symptoms and craving.

In compositions of this invention, focusing on the anabasine content per recommended dose the range of the amount of anabasine is between about 0.2 mg to about 8 mg, more preferably from about 0.5 mg to about 4 mg. Thus, for example, if a recommended daily dose ranges up to 8 oral films, or chewing gum pieces, then a person could be receiving from 0.2 mg to 64 mg/day, but most preferably between 2-16 mg/day of anabasine contained in a standardized extract.

Compositions of the invention preferably have from only small, or trace, amounts of nicotine or no nicotine at all. Thus, the amount of nicotine per recommended dose will be from 0 wt. % to trace levels (unless a product is formulated with explicit amounts of nicotine plus the herbal component as an aid for nicotine cessation or reduction).

When formulated as lozenges, chewing gums, or the other forms discussed above and below, it is contemplated that the herbal component will be present in an amount from about 5 mg to about 600 dry weight, or about 5 mg to 600 mg liquid extract. Such compositions will typically also include additional components such as a binder, a humectant, and flavoring agents such as sweeteners, artificial or natural fruit flavors, oils, and the like. Coloring may also be included. Different strategies for delivering the active principles will ntail different formulations and components specific to th se products.

Thus, to give an example, in one embodiment, the composition is includ d in a chewing gum formulation. The formulations of chewing gum are conventional, and well known to those skilled in the art. For example, a carri r may be provided that may be mixed with the herbal component. Suitable carriers, particularly in formulating chewing gums, comprise Arabic, guar, and natural rubber gums. Other typical components are sweeteners (sugar, saccharin, sorbitol, aspartame), flavoring agents (e.g., mints, fruits, spices), coloring agents, and the like.

For example, the chewing gum or solid carrier may be composed, in its basic formula, of ingredients such as sucrose, corn syrup, gum base, coloring and flavoring. Ingredients such as HSH (hydrogenated starch hydrolysate), sorbitol, xylitol, and/or isomalt can replace sucrose and corn syrup at different ratios. As an example of preparation, to a hot water jacketed stainless steel gum mixer equipped with sigma tangential blades rotating at 9-12 rpm with a 1:2 rotating ratio, molten gum base may be added at approximately 55-55EC, and corn syrup or HSH, added at room temperature in the desired amounts, and mixed until fully dispersed. When a homogeneous mix is obtained, sucrose or sorbitol, xylitol, or isomalt may be added, all in powder form, and mixed until fully dispersed. During the process of the addition of the powder material, the herbal component may be added. Color, flavoring, and any other ingredient deemed necessary for the particular formula may be added. The gummy mass is th n discharged from the gum mixer and conveyed to the gum forming equipment.

Thus, for example, the solid portion or chewing gum used as a carrier for th herbal component may be compos d of sucros (10-80%, preferably 15-50%), corn syrup (5-60%, preferably 10-30%), gum base (10-90%, preferably 20-80%), sorbitol (10-60%, preferably 20-50%), hydrogenated starch hydrolysat (HSH) (5-60%, preferably 10-30%), hydrolyzed proteins (1-8%, preferably 1.5-3.0%), isomalt (10-80%, preferably 15-50%), xylitol (10-80%, preferably 15-50%), artificial sweeteners (0.2-2.0%, preferably 0.5-1.0%), natural sweeteners, coloring, and flavor ingredients—to appearance and taste. Additional ingredients may include other botanical extracts, gelatin, glycerin, starch and modified starches (1-7%, preferably 1.5-5.0%), these being used for the purpose of modifying texture and chewing properties of the gum as w ll as to enhance the release of nicotine agonists from the gum matrix. The texture and physical properties of the finished product are affected by the final form of th chewing gum, which can also be in sugar or sugar-free form. Such a chewing gum formulation may also include a liquid center in the gum. In such case, th herbal component, preferably in the form of an herbal extract in suitable s lvent, may be incorporated into or serve as the liquid center.

In another embodiment, the herb or herbal extract component of this invention is included in sucking candies, syrups, oral films, intra-oral sprays, sub-lingual liquids, oral fast dissolving tablets for sub-lingual dispersion, micro-emulsions, sublingual buccal effervescents, trans-mucosal delivery sytems, lozenge formulations, all formulated for oral administration of the medication with local effects and absorption in the oral cavity. Known agents, binders, and the like as carriers may be used in such formulations.

Furth r, liquid preparations (wher th carri r is a liquid) and emulsions are also contemplated for the inventiv compositions to nabl oral mucosal dispersion and absorption.

It is to be understood that while the invention has been described above in conjunction with preferred specific embodiments, the description and examples are intended to illustrate and not limit the scope of the invention.

We claim:

1. A composition useful in relieving withdrawal symptoms and acute and later craving in a nicotine dependent/habituated person who is abstaining from or reducing nicotine intake, comprising:
an herb or an herbal extract providing one or more naturally occurring nicotine agonists, one of the nicotine agonists potentially being anabasine in an amount of at least about 0.1 (one tenth of one percent) weight percent of th herb or herbal extract, or 0.2 mg of anabasine or more per dose, the herb or herbal extract having from about 0 weight percent nicotine to a trace am unt (unless explicitly formulated as a combination of nicotine in some explicit quantity complexed with the herbal component as described herein); and
a carrier formulated for oral mucosal absorption (e.g., chewing gums, sucking candies, syrups, oral films, intra-oral sprays, sub-lingual liquids, oral fast dissolving tablets for sub-lingual dispersion, micro-emulsions, sublingual buccal effervescents, trans-mucosal delivery systems, lozenge formulations, and th like) of the active principles contained in the herb or herbal extract.
